(19) United States
(12) Patent Application Publication
(10) Pub. No.: US 2021/0375430 A1
(43) Pub. Date: Dec. 2, 2021

(54) METHODS, SYSTEMS, AND DEVICES FOR GENERATING A REFRESHMENT INSTRUCTION SET BASED ON INDIVIDUAL PREFERENCES

(71) Applicant: KPN INNOVATIONS, LLC., LAKEWOOD, CO (US)

(72) Inventor: Kenneth Neumann, LAKEWOOD, CO (US)

(21) Appl. No.: 17/032,080

(22) Filed: Sep. 25, 2020

Related U.S. Application Data
(63) Continuation-in-part of application No. 16/887,388, filed on May 29, 2020, now Pat. No. 10,861,076.

(51) Int. Cl. G16H 20/60; G06Q 10/10; G16H 50/20

(52) U.S. Cl. CPC G16H 20/60; G16H 50/20; G06Q 10/1095

(57) ABSTRACT

A system for generating a refreshment instruction set is disclosed. The system comprises a computing device configured to receive, from a remote device a user selection identifying a nourishment and health condition of user. Computing device determines an alimentary style relating to the user selection. An alimentary style classifier is used where alimentary style classifier inputs the nourishment and outputs an alimentary style. Computing device retrieves a plurality of recipes relating to the alimentary style and classifies the plurality of recipes based on the health condition. Computing device receives the plurality of recipes and, using training data and classification algorithm, a health alimentary classifier is generated. Based on the health condition and the health alimentary classifier, computing device generates refreshment instruction set. Computing device outputs the refreshment instruction and assigns constructed refreshment on a day of the appointment. A method for generating a refreshment instruction set is disclosed.

### CROSS-REFERENCE TO RELATED APPLICATIONS

This continuation-in-part application claims the benefit of priority of U.S. Non-Provisional patent application Ser. No. 16/887,388 filed on May 29, 2020 and entitled “METHODS, SYSTEMS, AND DEVICES FOR GENERATING A REFRESHMENT INSTRUCTION SET BASED ON INDIVIDUAL PREFERENCES”, which is incorporated by reference herein in its entirety.

### FIELD OF THE INVENTION

The present invention generally relates to the field of nourishment. In particular, the present invention is directed to methods, systems, and devices for generating a refreshment instruction set based on individual preferences.

### BACKGROUND

Systems that generate individual refreshment instructions are often overloaded with data. Frequently, individual preferences are analyzed and assessed on a massive scale. Worse, variants among selections and individual likes and dislikes regarding nourishment are not considered.

### SUMMARY OF THE DISCLOSURE

In an aspect, a system for generating a refreshment instruction set is disclosed. The system comprises a computing device configured to receive, from a remote device a user selection identifying a nourishment and a health condition of a user. Computing device determines an alimentary style relating to the user selection. An alimentary style classifier is used where the alimentary style classifier inputs the nourishment and outputs the alimentary style. Computing device retrieves a plurality of recipes relating to the alimentary style and classifies the plurality of recipes based on the health condition. Computing device receives the plurality of recipes and, using training data that correlates the health condition to the plurality of recipes and a classification algorithm, generates a health alimentary classifier. Based on the health alimentary classifier and the health condition, computing device generates a refreshment instruction set. Computing device outputs the refreshment instruction set by identifying an appointment relating to the user based on the schedule of the user. Computing device locates a constructed refreshment contained within the refreshment instruction set and assigns the constructed refreshment on a day of the appointment.

In another aspect, a method of generating a refreshment instruction set is disclosed. The method comprises receiving, from a computing device, a user selection identifying a nourishment and a health condition of a user. Computing device determines an alimentary style relating to the user selection. An alimentary style classifier is used where the alimentary style classifier inputs the nourishment and outputs the alimentary style. Computing device retrieves a plurality of recipes relating to the alimentary style and classifies the plurality of recipes based on the health condition. Computing device receives the plurality of recipes and, using training data that correlates the health condition to the plurality of recipes and a classification algorithm, a health alimentary classifier is generated. Based on the health condition and the health alimentary classifier, computing device generates a refreshment instruction set. Computing device outputs the refreshment instruction set by identifying an appointment relating to the user based on the schedule of the user. Computing device locates a constructed refreshment contained within the refreshment instruction set and assigns the constructed refreshment on a day of the appointment.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for generating a refreshment instruction set based on individual preferences;

FIG. 2 is a block diagram illustrating an exemplary embodiment of a selection database;

FIG. 3 is a representative diagram illustrating an exemplary embodiment of user selection data;

FIG. 4 is a process flow diagram illustrating an exemplary embodiment of a method of generating a refreshment instruction set based on individual preferences; and

FIG. 5 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to methods, systems, and devices for generating a refreshment instruction set based on self-reported health conditions. In an embodiment, a computing device receives a user selection relating to nourishment and a health condition. A computing device selects a plurality of recipes relating to a user's identified alimentary style by using a machine-learning classifier, where the classifier inputs nourishment and health conditions and outputs an alimentary style. The plurality of recipes is classified by a health alimentary classifier that uses training data correlating the health condition to the plurality of recipes which generates a refreshment instruction set. A constructed refreshment is outputted to the user on a day of an appointment based on the schedule of the user.

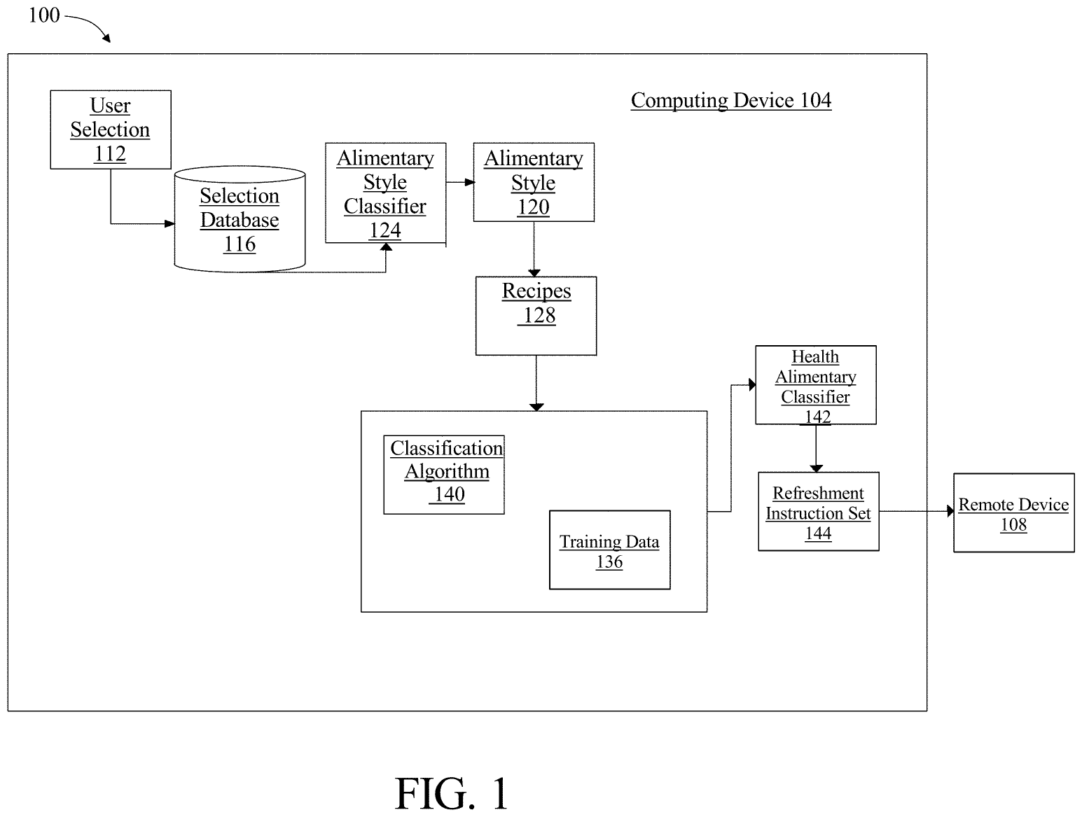

Referring now to FIG. 1, an exemplary embodiment of a system 100 for generating a refreshment instruction is illustrated. System 100 includes a computing device 104. Computing device 104 may include any computing device 104 as described in this disclosure, including without limitation a microcontroller, microprocessor, digital signal processor (DSP) and/or system on a chip (SoC) as described in this disclosure. Computing device 104 may include, be included in, and/or connect with a mobile device such as a mobile telephone or smartphone. Computing device 104 may include a single computing device 104 operating independently or may include two or more computing device 104 operating in concert, in parallel, sequentially or the like; two or more computing devices 104 may be included together in a single computing device 104 or in two or more computing devices 104. Computing device 104 may interface or connect with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an association, a building, a campus or other relatively small geographic space), a telephone network, a data network associated with a telephone/voice provider (e.g., a mobile communications provider data and/or voice network), a direct connection between two computing devices 104, and any combinations thereof. A network may employ a wired and/or a wireless mode of communication. In general, any network topology may be used. Information (e.g., data, software etc.) may be transmitted to and/or from a computer and/or a computing device 104. Computing device 104 may include but is not limited to, for example, a computing device 104 or cluster of computing devices 104 in a first position and a second computing device 104 or cluster of computing devices 104 in a second position. Computing device 104 may include one or more computing devices 104 dedicated to data storage, security, dispersal of traffic for load balancing, and the like. Computing device 104 may distribute one or more computing tasks as described below across a plurality of computing devices 104 of computing device 104, which may operate in parallel, in series, redundantly, or in any other manner used for dispersal of tasks or memory between computing devices 104. Computing device 104 may be implemented using a “shared nothing” architecture in which data is cached at the operative, in an embodiment, this may enable scalability of system 100 and/or computing device 104.

Continuing to refer to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence recurrently until a desired or commanded outcome is achieved; repetition of a step or a sequence of steps may be performed iteratively and/or recursively using outputs of previous repetitions as inputs to subsequent repetitions, assembling inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, computing device 104 is configured to receive from a remote device 108 a user selection 112 relating to nourishment and a health condition of a user. A remote device 108 includes any additional computing device, such as a mobile device, laptop, desktop, computer, and the like. A remote device 108 may include without limitation, a display in communication with computing device 104, where a display may include any display as described herein. Computing device 104 receives a user selection 112 from remote device 108 utilizing any network methodology as described herein.

With continued reference to FIG. 1, a “user selection” as described in this disclosure, is data describing a user's preference in regards to any source of nourishment, including any food and/or beverage consumed by a human being. A user selection 112 may indicate a user's like or dislike of an ingredient, a meal, a drink or beverage, and the like. For instance and without limitation, a user selection 112 may indicate that a user likes ingredients such as avocado, salmon, and jasmine rice, but the user dislikes black olives. In yet another non-limiting example, a user selection 112 may indicate that a user likes meals that include chicken alfredo, chicken parmesan, and spaghetti and meatballs, but the user dislikes meals that contain fish including fish tacos and pan sautéed cod. A user selection 112 may indicate a user's eating patterns including the number of meals a user eats each day, the times of the day the user prefers to eat meals, meals a user skips or does not eat, number of snacks a user consumes each day and the like. A user selection 112 may indicate a user's cooking and meal preparation patterns, including if a user cooks meals at home, orders meal preparation kits, orders prepared foods, shops for groceries online or in person at a grocery store, eats at restaurants, and the like. A user selection 112 may indicate a user's meal and ingredient source, such as if a user prefers ingredients that do not contain genetically modified organisms (GMOs), if a user prefers seafood that is wild caught and sustainable, if a user prefers free-range poultry, and/or if a user prefers organically sourced produce for example. Information pertaining to a user selection 112 may be stored in a selection database 116. Selection database 116 may be implemented, without limitation, as a relational database, a key-value retrieval datastore such as a NOSQL database, or any other format or structure for use as a datastore that a person skilled in the art would recognize as suitable upon review of the entirety of this disclosure.

With continued reference to FIG. 1, a “health condition” as used in this specification is a medical, mental, environmental, and/or psychological condition that may affect an otherwise healthy individual. The health condition may be known to the user as a result of a diagnoses by a medical professional. The health condition may be known to the user as a result of symptoms experienced by the user where the user researches the symptoms using tools available, for example, on the Internet, to match symptoms to a particular condition. The health condition may be a genetic condition or an inheritance condition where, for example, the user inherits an altered or changed gene from the parents. Examples of such genetic or inherited conditions may include, but not limited to cystic fibrosis, muscular dystrophy, and the like.

With continued reference to FIG. 1, computing device 104 is configured to determine an alimentary style 120 relating to a user selection 112. An “alimentary style,” as used in this disclosure, is data describing a user's eating style and/or eating habits. An alimentary style 120 may describe a particular diet that a user may follow, such as a user who consumes meat, fish, eggs, vegetables, fruits, nuts, seeds, herbs, spices, health fats and oils and avoids processed foods, sugar, soft drinks, grains, dairy products, legumes, artificial sweeteners, vegetable oils, and trans-fat may be identified as having an alimentary style 120 of a paleo style. In yet another non-limiting example, a user who limits the consumption of foods containing short-chain carbohydrates including fermentable oligosaccharides, disaccharides, monosaccharides, and polyols (FODMAP) may be identified as having an alimentary style 120 of a low-FODMAP style. Computing device 104 determines an alimentary style 120 utilizing a user refreshment record. Computing device 104 is configured to receive from a remote device an element of a user refreshment record. An “element of a user refreshment record,” as used in this disclosure, is any historical records of meals and/or ingredients that a user previously consumed. An element of a user refreshment data may include any meal journals, tracked meals, recorded meals, descriptions of previously consumed meals, photographs and/or pictures of meals and the like that contain any information describing any meal and/or ingredient that a user previously consumed. In an embodiment, information pertaining to an element of a user refreshment record may be stored within selection database 116. For instance and without limitation, an element of a user refreshment record may describe a user's lunch from the previous day which included a turkey sandwich served on multi-grain bread with lettuce, tomato, pickles, and mustard along with a side salad. An element of a user refreshment record may contain a timestamp, indicating the day and/or time that a user consumed a particular meal. For instance, and without limitation, an element of a user refreshment record may contain a meal diary containing entrees of every meal that a user ate over a two week period, containing the day, time, and meal that was consumed. Computing device 104 determines an alimentary style 120 utilizing an element of a user refreshment record. For instance and without limitation, an element of a user refreshment record that contains meals that do not contain any meat or fish containing meals but that does contain entrees containing eggs and cheese products may be identified as an alimentary style of a vegetarian. In yet another non-limiting example, an element of a user refreshment record that contains meals that vegetables, fruits, nuts, seeds, legumes, potatoes, whole grains, breads, herbs, spices, fish, seafood, and olive oil may be identified as an alimentary style 120 that follows the Mediterranean style of eating. Information pertaining to an alimentary style 120 may be contained within recipe database. Computing device 104 may utilize information contained within recipe database to determine an alimentary style 120. Recipe database may be implemented as any data structure suitable for use as selection database 116 as described above in more detail. In an embodiment, an alimentary style 120 may indicate one or more foods that a user chose not to consume because of an allergy or an intolerance. For instance and without limitation, an alimentary style 120 may indicate that a user eats all foods except the user does not consume any foods and/or meals that contain dill weed, as the user dislikes the taste of dill.

With continued reference to FIG. 1, computing device 104 identifies an alimentary style 120 using an alimentary style classifier 124. A “classification algorithm,” as used in this disclosure, is a process whereby a computing device 104 sorts inputs not categories or bins of data. Classification algorithms may include linear classifiers such as logistic regression, Naive Bayes classification, Fisher's linear discriminant, k-nearest neighbors, support vector machines, quadratic classifiers, Kernel estimation, decision trees, boosted trees, random forest, neural networks, and the like. An “alimentary style classifier,” as used in this disclosure, is a classification algorithm that utilizes a user selection relating to nourishment as an input, and outputs an alimentary style relating to the user selection. Computing device 104 trains a classification algorithm utilizing training data, including any of the training data as described herein. Training data may be obtained from previous iterations of generating a classification algorithm, user inputs and/or user surveys, expert input, and the like. Computing device 104 trains alimentary style classifier utilizing training data, wherein training data includes a plurality of data entries containing user selections correlated to corresponding alimentary styles. In one embodiment, computing device 104 may be configured to train the alimentary style classifier 124 using training data where the training data uses health condition of the user as an input and outputs a corresponding alimentary style. Training data contains correlations that alimentary style classifier may use to model relationships between two or more categories of data elements. For instance, and without limitation, training data may include a plurality of data entries, each entry representing a set of data elements that were recorded, received, and/or generated together; data elements may be correlated by shared existence in a given data entry, by proximity in a given data entry, or the like. Multiple data entries in training data may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data according to various correlations; correlations may indicate causative and/or predictive links between categories of data elements, which may be modeled as relationships such as mathematical relationships by machine-learning processes as described in further detail below. Training data may be formatted and/or organized by categories of data elements, for instance by associating data elements with one or more descriptors corresponding to categories of data elements. As a non-limiting example, training data may include data entered in standardized forms by persons or processes, such that entry of a given data element in a given field in a form may be mapped to one or more descriptors of categories. Elements in training data may be linked to descriptors of categories by tags, tokens, or other data elements; for instance, and without limitation, training data may be provided in fixed-length formats, formats linking positions of data to categories such as comma-separated value (CSV) formats and/or self-describing formats such as extensible markup language (XML), enabling processes or devices to detect categories of data.

With continued reference to FIG. 1, computing device 104 is configured to retrieve a plurality of recipes 128 relating to an alimentary style 120. A “recipe,” as used in this disclosure, is a set of instructions for the preparation of a particular dish, including a list of ingredients required to prepare the dish. A recipe relates to an alimentary style 120, when the ingredients require to prepare the dish comply with the alimentary style 120. For instance, and without limitation, a recipe for orecchiette pasta with broccoli sauce relates to a vegetarian alimentary style 120, while a recipe for a filet mignon served with potatoes does not relate to a vegetarian alimentary style 120. In an embodiment, a recipe may relate to one or more alimentary style 120. For example, a recipe for coffee crusted steak served with green beans and mashed sweet potatoes may relate to a paleo alimentary style 120, a gluten free alimentary style 120, a low-FODMAP alimentary style 120, a dairy free alimentary style 120, a grain free alimentary style 120 and the like. Information pertaining to recipes and alimentary style 120 may be stored within selection database 116. Computing device 104 locates recipes relating to an alimentary style 120 utilizing user refreshment effort data. Computing device 104 identifies an element of data relating to a user refreshment effort. A “user refreshment effort,” as used in this disclosure, is any indication as to recipe complexity, cooking time, and/or culinary skill that a user prefers. Information pertaining to a user refreshment effort may be contained within selection database 116. For instance and without limitation, a user refreshment effort may indicate that a user dislikes cooking and/or preparing any meals that take longer than thirty minutes to prepare. In yet another non-limiting example, a user refreshment effort may indicate that a user enjoys preparing complex recipes on the weekends when the user has more time and isn't at work all day, versus during the week the user only likes to prepare meals that take fifteen minutes or less. In yet another non-limiting example, a user refreshment effort may indicate that a user only likes to cook no more than three meals each week. Computing device 104 locates recipes intended for a user refreshment effort. In an embodiment, recipes may be stored within a recipe database by how much effort they require to prepare, seasonality of ingredients contained within recipes, cooking times, effort and/or skill level needed to prepare, and/or information pertaining to alimentary style 120. In such an instance, computing device 104 matches information relating to a user refreshment effort to information stored about a recipe. For instance and without limitation, computing device 104 locates a user refreshment effort that indicates a user enjoys cooking complex meals because the user attended culinary school, and as such computing device 104 locates recipes that are complex and require greater skill and effort to prepare. In yet another non-limiting example, computing device 104 may generate a query to locate recipes contained within selection database 116 to locate recipes that comply with a user refreshment effort. A “query,” as used in this disclosure, is any information utilized to identify and/or select a alimentary style 128 from selection database 116. For instance and without limitation, a user refreshment effort that describes a user who enjoys cooking meals that take one hour or less to prepare and that are gluten free may be utilized to generate a query to identify recipes that can be cooked in one hour or less and that contain ingredients that are gluten free.

Still referring to FIG. 1, computing device 104 may be configured to classify the plurality of recipes 128 as a function of the health condition. Health alimentary classifier 132 is generated by using training data 136 correlating the health condition to the plurality of recipes 128 and a classification algorithm 140 as described herein. Computing device 104 receives the plurality of recipes 128 and generates a refreshment instruction set 144 as a function of the plurality of recipes 128 and the health condition. A “refreshment instruction set,” as used in this disclosure, is a meal plan containing recommended refreshments and a suggested temporal attribute. An health alimentary classifier 132. as used in this disclosure, is a classification algorithm that utilizes a user selection relating to health condition of the user and the plurality of recipes 128 as inputs, and outputs an refreshment instruction set 144 relating to the health condition of the user. Computing device 104 trains a classification algorithm utilizing training data, including any of the training data as described herein. A “recommended refreshment,” as used in this disclosure, is any recommended meals contained within a refreshment instruction set 144. A meal plan may include one or more recommended meals assigned to a particular day, time, and/or meal. For instance and without limitation, a meal plan may contain a week's worth of suggested breakfasts, lunches, dinners, and/or snacks for a user to consume throughout the week. A “refreshment,” as used in this disclosure, is any meal, snack, beverage, drink, sub-part of a meal, spice, nutritional supplement and the like intended for consumption by a human being. A “temporal attribute,” as used in this disclosure, is any recommended day, time, and/or meal that a refreshment is recommended to be consumed at. A temporal attribute may include a specific mealtime, such as a refreshment that is recommended to be consumed for lunch at 12:30 pm, or a refreshment that is recommended to be consumed for breakfast at 7:00 am on a Wednesday. Computing device 104 generates a refreshment instruction set 144 utilizing a plurality of recipes 128 relating to an alimentary instruction set and assigns a recipe to a various day, time, and/or meal. For instance and without limitation, computing device 104 may assign a refreshment option of millet cereal topped with coconut cream and fresh berries to breakfast on a Monday, and a refreshment option of dairy free eggplant parmesan to dinner on a Tuesday, for a user following a vegan alimentary style 120. Computing device 104 may select a particular recipe for a particular day, meal, and/or time, by obtaining information relating to a user's calendar. Computing device 104 identifies an appointment relating to a user. An “appointment,” as used in this disclosure, is an event contained within a schedule. An appointment may include a work-related engagement, such as a meeting a user may have with a co-worker. An engagement may include a personal engagement, such as an appointment a user may have with the user's medical doctor. Computing device 104 may retrieve information relating to an appointment for a user from selection database 116. In an embodiment, computing device 104 receives inputs from remote device operated by a user containing any calendar information that contains information relating to a user's appointments.

With continued reference to FIG. 1, computing device 104 is configured to output refreshment instruction set 144. Computing device 104 may identify an appointment relating to the user based on the schedule of the user. A “schedule,” as used in this disclosure, is a chart containing information pertaining to any appointments scheduled throughout a day. An appointment may include any personal and/or work-related event that may take up any part of a user's day. For instance and without limitation, an appointment may include time spent commuting to and from work each day. In yet another non-limiting example, an appointment may include a work related gala that a user has to partake in one night after work from 6 pm-10 pm. In yet another non-limiting example, an appointment may include a doctor's appointment that a user has. Computing device 104 receives a schedule from a remote device utilizing any network methodology as described herein.

With continued reference to FIG. 1, computing device 104 locates a constructed refreshment contained within a refreshment instruction set 144 and assigns the constructed refreshment on the day of the appointment. A “constructed refreshment,” as used in this disclosure, is a prepared meal that requires minimal cooking and/or preparation time. A constructed refreshment may include a meal that was previously prepared and/or cooked and requires very minimal preparation and/or hands on cooking time by a user. For example, a constructed refreshment may include a previously cooked meal that a user needs to reheat in an oven to serve. In yet another non-limiting example, a constructed refreshment may include a meal that requires very little cooking time and/or preparation such as making a sandwich with cold cuts or preparing quick cooking oats that are ready in five minutes. Computing device 104 assigns a constructed refreshment on the day of an appointment. In an embodiment, computing device 104 may assign a constructed refreshment when an appointment may be scheduled within a certain time frame of when a user typically consumes meals. For example, a constructed refreshment such as a yogurt parfait that can be prepared within mere minutes may be assigned on a day when a user has an appointment at 9 am and the user usually eats breakfast at 8:30 am. Information pertaining to a user's typical eating schedule and mealtimes may be contained within selection database 116.

With continued reference to FIG. 1, computing device 104 is configured to receive from a remote device a user location. A “user location,” as used in this disclosure, is a description of any geographical location where a user is presently located and/or intendeds to be located at a future date and/or time. A user location may include a global positioning system (GPS) of a user, including for example, the GPS location that may be obtained from remote device. A user location may include a description of the latitude and longitude of a position where a user is currently located and/or a position where a user may be located in the future. Computing device 104 identifies offered elements contained within a user location. An “offered element,” as used in this disclosure, is any recipe ingredient available for sale and/or purchase within the user location. Information pertaining to an offered element may be contained within a recipe database. A recipe database may be implemented as any data structure suitable for use as selection database 116 as described above in more detail. An offered element may be available for sale and/or purchase at any online and/or retail store location such as for example a grocery store, supermarket, bodega, corner store, food mart, food store, market, and the like. Information pertaining to availability of offered element available at online and/or retail store locations may be updated and stored within recipe database. Such information may be received by computing device 104 utilizing any network methodology as described herein. For example, an offered element such as fresh rhubarb may only be available during certain times of the year based on a user's location, such as in Colorado where rhubarb is only available from late June through early July, while in California rhubarb may be available from November through June. In yet another non-limiting example, an offered element may only be available to be acquired and/or purchased in certain areas of the country, such as Wellfleet Oysters that may only be available from Wellfleet, Massachusetts and may only be sold in neighboring states throughout New England. Computing device 104 adjusts a recipe utilizing offered element contained within a user location and a user alimentary style 120. Computing device 104 adjusts a recipe such as by suggesting a second ingredient that can be substituted in place of a first ingredient that may not be available. For example, computing device 104 may identify an offered element such as hazelnuts that may be available in the Pacific Northwest and may be available in abundance during peak harvesting season within the Pacific Northwest. In such an instance, computing device 104 may adjust a recipe that contains an ingredient such as walnuts, and instead suggests hazelnuts instead, when they are available during peak season. Information pertaining to ingredients and offered element that can be substituted may be contained within the recipe database.

With continued reference to FIG. 1, computing device 104 is configured to receive a user refreshment activity from remote device. A “refreshment activity,” as used in this disclosure, is a description of any action and/or steps that a user took in response to a refreshment instruction set 144. A refreshment activity may include any ingredients purchased and/or necessary to prepare one or more recipes contained within a refreshment instruction set 144. A refreshment activity may include an action that a user took, such as to prepare and cook one or more recipes contained within a refreshment instruction set 144. A refreshment activity may include a log of one or more meals that a user consumed. Computing device 104 may classify, using a first classification algorithm, a user refreshment activity to an adherence label. A “first classification algorithm,” as used in this disclosure, is a process whereby computing device 104 derives from training data, a model known as a “classifier” for sorting inputs into categories or bins of data. Classification algorithms may include linear classifiers such as logistic regression, Naïve Bayes classification, Fisher's linear discriminant, k-nearest neighbors, support vector machines, quadratic classifiers, Kernel estimation, decision trees, boosted trees, random forest, neural networks, and the like. A first classification algorithm utilizes a user refreshment activity as an input, and outputs an adherence label. A classifier may be trained using training data. “Training data,” as used in this disclosure, is data containing correlations that a machine-learning process including a machine-learning algorithm and/or machine-learning process may use to model relationships between two or more categories of data elements. Training data may be formatted to include labels, for instance by associating data elements with one or more descriptors corresponding to categories of data elements. Training data may not contain labels, where training data may not be formatted to include labels. Training data may be obtained from records of previous iterations of generating first classification algorithm, user inputs, user questionnaire responses, expert inputs, and the like.

With continued reference to FIG. 1, an “adherence label,” as used in this disclosure, is a description as to how compliant a user has been with a refreshment instruction set 144. An adherence label may indicate compliance on a continuum and may reflect a user's compliance with numerical and/or character scores. For example, adherence may be calculated using numerical scores on a scale from 0 to 10, where an adherence label containing a score of 0 may indicate a user who was completely noncompliant, while an adherence label containing a score of 10 may indicate a user who was completely compliant. In yet another non-limiting example, compliance may be reflected with character scores, where an adherence label containing a response of extremely compliant may be used to describe a user who followed all recommendations contained within a refreshment instruction set 144, while an adherence label containing a response of not compliant may be used to describe a user who did not follow any recommendations contained within a refreshment instruction set 144. Computing device 104 updates a refreshment instruction utilizing an adherence label. For example, an adherence label that indicates a user was not compliant and did not follow any recommendations contained within a refreshment instruction set 144, may be utilized to select recipes that are very easy to prepare, and that do not require complex ingredients. In yet another non-limiting example, an adherence label 156 that indicates a user was mostly compliant may be utilized by computing device 104 to recommend similar meals and/or similar ingredients in an updated refreshment instruction set 144.

With continued reference to FIG. 1, computing device 104 is configured to create a list of elements necessary to create a plurality of recommended refreshments. A “list,” as used in this disclosure, is any aggregation of ingredients needed to prepare a refreshment instruction set 144. A list may be categorized based on elements offered and/or available for purchase at refreshment providers. For example, information contained within selection database 116 may contain preferences as to grocery stores and/or e-commerce sites where a user may purchase ingredients. In such an instance, computing device 104 categorizes elements from a list, to aggregate items that may be available and/or located together at a certain refreshment provider. For instance and without limitation, selection database 116 may contain information that a user purchases all meat ingredients from a butcher and all produce ingredients, and pantry items from a grocery store, and all dairy products from a local dairy farm. In such an instance, computing device 104 categorizes a list so that there is a first list for all ingredients a user needs to purchase from a butcher, a second list for all ingredients a user needs to purchase from a grocery store, and a third list for all ingredients a user needs to purchase from a local dairy farm.

With continued reference to FIG. 1, computing device 104 is configured to generate a recipe machine-learning model that utilizes a user selection 112 as an input and outputs recommended refreshments. A “recipe machine-learning model,” as used in this disclosure, is a mathematical representation of a relationship between inputs and outputs, as generated using any machine-learning process and/or machine-learning algorithm including without limitation any process as described herein, and stored in memory; an input is submitted to a machine-learning model once created, which generates an output based on the relationship that was derived. Generating recipe machine-learning model may include calculating one or more supervised machine-learning algorithms including active learning, classification, regression, analytical learning, artificial neural network, backpropagation, boosting, Bayesian statistics, case-based learning, genetic programming, Kernel estimators, naive Bayes classifiers, maximum entropy classifier, conditional random field, K-nearest neighbor algorithm, support vector machine, random forest, ordinal classification, data pre-processing, statistical relational learning, and the like. Generating recipe machine-learning model may include calculating one or more unsupervised machine-learning algorithms, including a clustering algorithm such as hierarchical clustering, k-means clustering, mixture models, density based spatial clustering of algorithms with noise (DBSCAN), ordering points to identify the clustering structure (OPTICS), anomaly detection such as local outlier factor, neural networks such as autoencoders, deep belief nets, Hebbian learning, generative adversarial networks, self-organizing map, and the like. Generating recipe machine-learning model may include calculating a semi-supervised machine-learning algorithm such as reinforcement learning, self-learning, feature learning, sparse dictionary learning, anomaly detection, robot learning, association rules and the like. Recipe machine-learning model is trained by computing device 104 using training data, including any of the training data as described herein. Training data may be obtained from records of previous iterations of generating recipe machine-learning model, user inputs and/or questionnaire responses, expert inputs, and the like. Recipe machine-learning model may be implemented as any machine-learning process, including for instance, and without limitation, as described in U.S. Nonprovisional application Ser. No. 16/375,303, filed on Apr. 4, 2019, and entitled “SYSTEMS AND METHODS FOR GENERATING ALIMENTARY INSTRUCTION SETS BASED ON VIBRANT CONSTITUTIONAL GUIDANCE,” the entirety of which is incorporated herein by reference. Recipe machine-learning model is trained using training data to select recommended refreshments favored by a user selection 112. In an embodiment, user selection 112 contained within selection database 116 may be utilized as training data to customize and train recipe machine-learning model individually for each user. For instance and without limitation, user selection 112 that indicate a user prefers to eat foods that contain protein choices that contain either chicken, tofu or salmon and the user dislikes protein choices that contain beef or pork may be utilized as training data to generate recommended refreshments such as chicken picada, tofu and green bean stir fry, and miso glazed salmon, and to not generate recommended refreshments such as a ground beef stir fry or a pork burger. In another embodiment, recipe machine-learning model may output a plurality of recommended refreshments as a function of the health condition of the user. For instance and without limitation, a user may have a gluten allergy. In this example, recipe machine-learning model may output recommended refreshment suitable for the user where the recommended refreshment are gluten-free.

With continued reference to FIG. 1, system 100 includes a remote device for generating a refreshment instruction set 144 based on individual preferences. Remote device includes any of the remote devices as described herein. Remote device includes a preference module, which may be implemented as any hardware and/or software module. Preference module is configured to collect a user selection 112 relating to nourishment. A user selection 112 includes any of the user selection 112 as described above in more detail. Preference module may collect a user selection 112 such as by displaying a photograph and/or names of one or more refreshments on remote device and collecting a user's likes and/or dislikes regarding refreshments. For instance and without limitation, preference module may display a photograph of a refreshment such as pasta and prompt a user to answer questions regarding pasta, such as if the user likes or dislikes pasta, how many times each week the user eats pasta, brands of pasta that the user likes or dislikes, serving size of pasta that the user likes, and the like. Remote device may include a graphical user interface that may display refreshments to a user. Graphical user interface may include without limitation, a form or other graphical element having display fields, where one or more elements of information may be displayed. Graphical user interface may include sliders that a user may adjust to indicate the user's like or dislike of various refreshments. Graphical user interface may include free form textual entries, where a user may type in information regarding user selection 112. In an embodiment, remote device may display on graphical user interface a series of questions to prompt a user for more information. In an embodiment, graphical user interface may display one or more answer choices that a user may selection an answer from in response to a questionnaire or other prompts for information. In yet another non-limiting example, a user may be able to type in a customized response in a free form textual entry. Information pertaining to a user selection 112 may be stored locally on remote device, such as in memory or in a database located on remote device. In an embodiment, information pertaining to a user selection 112 may be transmitted from remote device to computing device 104 to be stored within selection database 116.

With continued reference to FIG. 1, preference module located on remote device is configured generate a list of preferred elements. A “list of preferred elements,” as used in this disclosure, is any aggregation of refreshments including ingredients and/or meals that a user likes. Remote device assembles a list of preferred elements by evaluating a user selection 112, to identify refreshments that a user prefers. For instance and without limitation, remote device may evaluate a user selection 112 that suggests a user likes salmon, avocado, and wild rice, and dislikes baked potato, cod, and halibut to generate a list of preferred elements that contains salmon, avocado, and wild rice. In yet another non-limiting example, a user selection 112 that indicates a user habitually consumes turkey in a lettuce wrap with pickles for lunch may be utilized to generate a list of preferred elements. Remote device uses a list of preferred elements to create a refreshment instruction set 144. Refreshment instruction set 144 includes any of the refreshment instruction set 144 as described herein. Preference module is configured to generate refreshment instruction set 144 using one or more machine-learning processes, including any of the machine-learning processes as described herein. Preference module is configured to generate recipe machine-learning model, wherein recipe machine-learning model utilizes a user selection as an input, and outputs a plurality of recommended refreshment contained within refreshment instruction set. Preference module trains recipe machine-learning model utilizing training data, including any of the training data as described herein. Training data utilized to train recipe machine-learning module is obtained from previous iterations of recipe machine-learning model, user inputs and/or user questionnaires, and/or expert inputs. Remote device utilizes a list of preferred elements to generate a refreshment instruction set 144. For instance and without limitation, a list of preferred elements may contain ingredients that a user routinely consumes, such as bacon, steak, kale, spinach, oatmeal, berries, sweet potato, chicken, and gluten free bread. In such an instance, remote device utilizes preferred elements to generate a refreshment instruction set 144. In an embodiment, remote device may select other ingredients to include within refreshment instruction set 144 that may be related to and/or similar to ingredients that a user routinely consumes. For example, a list of preferred elements that indicates a user consumes ground beef may be utilized by remote device to recommend a refreshment contained within refreshment instruction set 144 that contains filet mignon or a ribeye steak. In yet another non-limiting example, a list of preferred elements that does not include any cruciferous vegetables such as cauliflower, Brussel sprouts, and/or kale may be utilized by remote device to generate a refreshment instruction set 144 that does not contain any cruciferous vegetables.

With continued reference to FIG. 1, remote device includes an image capture device designed and configured to capture an image of a refreshment. An “image capture device,” as used in this disclosure, is any device suitable to take a photograph of a refreshment. An image capture device may include for example, a camera, mobile phone camera, scanner, and the like. A user may utilize an image capture device located on remote device to take a photograph of a user selection 112 relating to nourishment. For example, a user may utilize an image capture device to photograph meals that a user enjoys eating, foods that a user likes, meals that a user dislikes, foods that a user dislikes, and the like. Remote device utilizes an image of a refreshment to generate a list of preferred elements as described above in more detail.

Still referring to FIG. 1, computing device 104 may be configured to transmit the refreshment instruction set to the remote device 103 and receive a user response where the user rejects the refreshment instruction set 144 received. For example, a user may not like the refreshment instruction set 144 or may prefer a different refreshment instruction set based on a desire for a different refreshment instruction set 144 received. The user may reject the instruction set 140 received. Computing device 104, upon receiving the rejection by the user of the rejection of the refreshment instruction set, may transmit a second refreshment instruction set to remote device 108. In another embodiment, computing device 104 may transmit a communication including information based on the health condition and the refreshment instruction set 144. As a non-limiting example, a low cholesterol refreshment instruction set received by the user based on the user having a diagnosis of hypercholesterolemia (high cholesterol) may also include information on the disease, other suggestions on how to manage the disease, exercise suggestions, changes on daily routine, and the like.

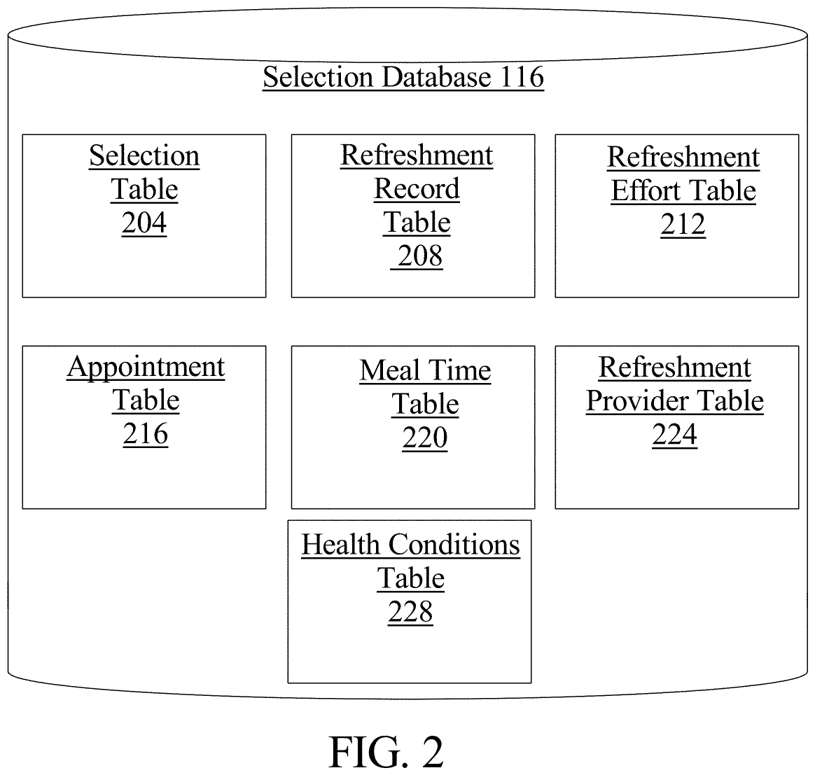

Referring now to FIG. 2, an exemplary embodiment of selection database 116 is illustrated. Selection database 116 may be implemented as any data structure as described above in more detail in reference to FIG. 1. One or more tables contained within selection database 116 may include selection table 204; selection table 204 may contain a user selection 112 relating to nourishment. For instance and without limitation, selection table 204 may contain an entry specifying that a user likes foods that taste sweet such as berries, watermelon, cantaloupe, and sweet potatoes, and the user dislikes foods that contain eggs. One or more tables contained within selection database 116 may include refreshment record table 208; refreshment record table 208 may include one or more elements of a user refreshment record. For instance and without limitation, refreshment record table 208 may contain an entry describing a meal a user consumed for dinner that consisted of a slow braised lamb shank served over Israeli couscous with a side of steamed carrots. One or more tables contained within selection database 116 may include refreshment effort table 212; refreshment effort table 212 may include elements of data relating to a user refreshment effort. For instance and without limitation, refreshment effort table 212 may contain an entry specifying that a user prefers to cook meal that can be ready in forty five minutes and that require no more than ten ingredients or less. One or more tables contained within selection database 116 may include appointment table 216; appointment table 216 may contain information pertaining to any appointments a user has. For instance and without limitation, appointment table 216 may contain an entry containing all appointments a user has over the next upcoming three weeks, including all personal appointments, work related appointments and the like. One or more tables contained within selection database 116 may include mealtime table 220; mealtime table 220 may include information describing times of the day when the user generally consumes various meals. For instance and without limitation, mealtime table 220 may contain an entry specifying that a user consumes breakfast every day at 7:30 am, lunch at 12:30 pm, dinner at 6:30 pm, and a snack at 3:30 pm on days when a user participates in a fitness class. One or more tables contained within selection database 116 may include refreshment provider table 224; refreshment provider table 224 may include information pertaining to a user's preferences regarding refreshment providers. For instance and without limitation, refreshment provider table 224 may contain an entry specifying refreshment providers that a user purchases refreshments from, such as a local farm stand where a user purchases fresh dairy and produce products, and a local grocery store where a user purchases all other refreshments including meat products, canned goods, pantry staples, and the like. One or more tables contained within selection database 116 may include a health conditions table 228; health conditions table 224 may include information pertaining to a user's health conditions. For instance, and without limitation, health conditions table 228 may contain conditions that were diagnosed by a health professional or health conditions that may be self-diagnosed by the user. Health conditions table 228 may contain historical information as to prior historical conditions reported by the user.

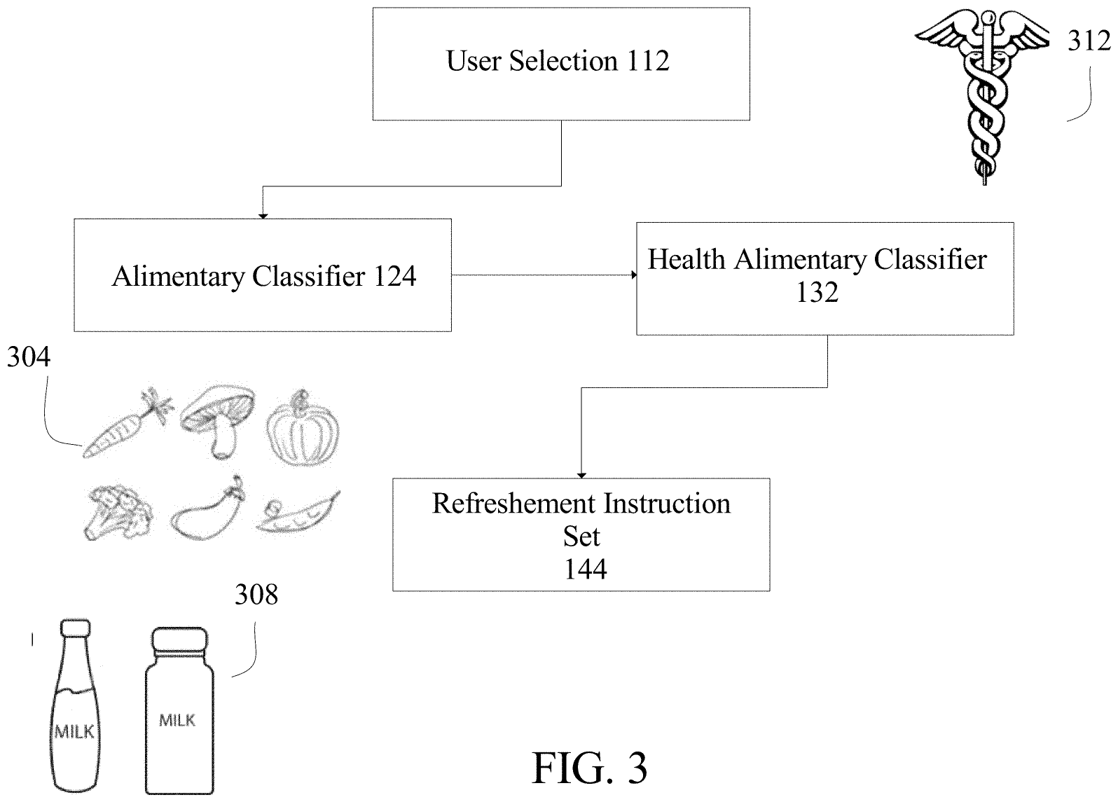

Referring now to FIG. 3, an exemplary embodiment of user selection 112 data that may be utilized to generate a refreshment instruction set 144 is illustrated. Computing device 104 utilizes information pertaining to a user selection 112 as an input to generate a machine-learning model. The machine-learning model outputs a plurality of recommended refreshments. Information pertaining to a user selection 112 may be stored within selection database 116 as described above in more detail in reference to FIG. 1. In a non-limiting example, a user selection 112 may relate to nourishment 304 such as, but not limited to vegetables, fruits, and the like. A user selection 112 may relate to nourishment including types of beverages 308 which may include, but not limited to dairy products, fruit juices, nut milk, and the like. A user selection 112 may relate to a user's preference regarding sourcing of ingredients such as if a user buys only organically grown products and ingredients, fair trade ingredients, non-genetically modified organisms (GMOs), and the like. A user selection 112 may relate to a user's habits, including how often the user shops for ingredients, refreshment providers that the user shops at, whether the user shops in person or online and the like. A user selection 112 may relate to a user's preference regarding ingredients, such as particular ingredients a user likes or dislikes, meals a user likes or dislikes, and the like. A user selection 112 may relate to a user's preference regarding prepared foods such as how many food from a restaurant if a user dines in at a restaurant or purchases carry-out or delivery and the like. Additionally, user selection 112 may include health conditions 312. Health conditions may include conditions related to nourishment. For example, a food allergy, or a user with an issue with food such as a lactose intolerant user. Health conditions may be unrelated to nourishment such as conditions caused by pathogens such as a virus or bacteria. Computing device 104 utilizes one or more user selection 112 as an input to a health alimentary classifier outputs a refreshment instruction set 144, containing a meal plan recommended for a user over a specified period of time.

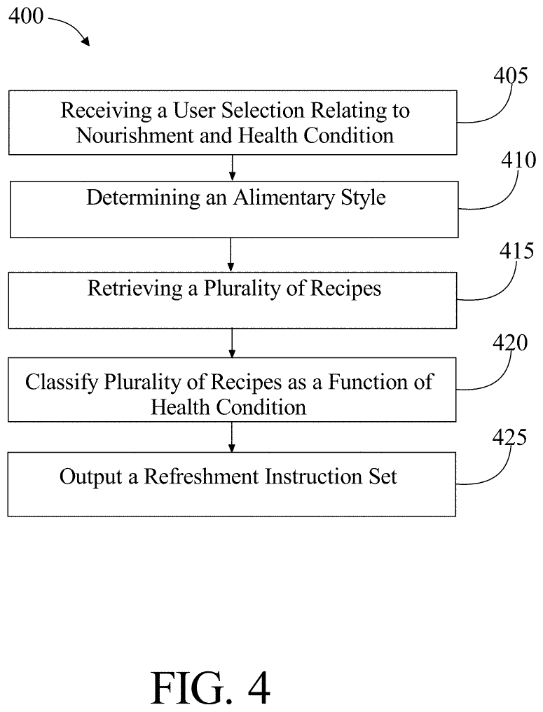

Referring now to FIG. 4, an exemplary embodiment 400 of a method of generating a refreshment instruction set 144 based on the health condition of an individual is illustrated. At step 405, computing device receives from a remote device, a user selection relating to nourishment and a health condition of the user. This may be implemented, without any limitations as described in FIGS. 1-3.

With continued reference to FIG. 4, at step 410, computing device determines an alimentary style relating to a user selection 112 using an alimentary style classifier that uses nourishment and health condition of the user and outputs an alimentary style 120. This step may be implemented, without limitations, as described in FIGS. 1-3. An alimentary style 120 describes a user's eating style and/or eating habits. An alimentary style 120 may describe a certain diet that a user may follow such as a lactose free diet, or a ketogenic diet. Information pertaining to an alimentary style 120 may be stored within recipe database. An alimentary style 120 may describe certain foods that a user does not consume because of an allergy, dislike for a particular food and the like. For example, an alimentary style 120 may describe a user who consumes a standard American diet but does not eat any pork products because the user dislikes the taste of all pork products. In yet another non-limiting example, an alimentary style 120 may describe a user who does not consume any products that contain tree nuts, because the user has a long-standing allergy to all tree nut containing products. Computing device 104 determines an alimentary style 120 by consulting alimentary database to identify a particular alimentary style 120 utilizing information contained within a user selection 112. Computing device 104 may generate a query and search alimentary database to locate an alimentary style 120 utilizing information contained within a user selection 112. For instance and without limitation, a user selection 112 that indicates a user dislikes all meat products may be utilized by computing device 104 to generate a query and locate an alimentary style 120 such as a pescatarian alimentary style 120 or a vegetarian alimentary style 120. Computing device 104 may ask a user a series of follow-up questions when more than one alimentary style 120 may apply. For example, in the previous example, computing device 104 may prompt a user to ask a series of questions such as if the user is comfortable consuming fish containing products or if the user eliminates all animal containing products.

Additionally or alternatively, and with continued reference to FIG. 4, computing device may train the alimentary style classifier using training data where the training data correlates a health condition to a corresponding alimentary style. This may be implemented, without limitation, as described in FIGS. 1-3.

With continued reference to FIG. 4, computing device determines an alimentary style 120 utilizing a user refreshment record. A user refreshment record includes any of the user refreshment record as described above in more detail in reference to FIG. 1. A user refreshment record may contain a description of one or more meals that a user may have previously consumed. For instance, and without limitation, a user refreshment record may contain an entry containing a description of all meals a user consumed over the previous seven days. Computing device 104 evaluates a user refreshment record to determine an alimentary style 120 utilizing information contained within the user refreshment record. For example, computing device 104 may analyze components of each meal contained within a user refreshment record to determine an alimentary style 120. Computing device 104 may consult recipe database to discover more information relating to alimentary styles that may be compiled based on expert inputs and expert advice. Computing device 104 determines an alimentary style 120 using an element of user refreshment data.

With continued reference to FIG. 4, at step 415, computing device retrieves a plurality of recipes relating to the alimentary style. This may be implemented, without limitation, as described in FIGS. 1-3. A recipe, is a set of instructions for the preparation of a particular dish, including a list of ingredients required to prepare the dish, as described above in more detail in reference to FIG. 1. A recipe relates to an alimentary style 120 when the recipe contains ingredients that conform with the alimentary style 120. For instance, and without limitation, a recipe for chicken parmesan would not relate to a vegan alimentary style 120, while a recipe for tofu and broccoli stir-fry would relate to a vegan alimentary style 120. In yet another non-limiting example, a recipe for steak frites may relate to a gluten free alimentary style 120 and a grain free alimentary style 120. Information pertaining to alimentary style 120 and recipes may be stored within recipe database as described above in more detail. Computing device 104 locates recipes utilizing data relating to a user refreshment effort. A user refreshment effort includes any of the user refreshment effort as described above in more detail in reference to FIG. 1. A user refreshment effort may contain a description of how much time, effort, and/or skill a user has in regard to preparing meals and/or following recipes. For example, a user refreshment effort may contain information describing a user's culinary skills and experience, which may reflect that the user enjoys cooking meals that take twenty minutes or less. In yet another non-limiting example, a user refreshment effort may describe a user who has taken adult education cooking classes and who enjoys cooking complex meals. Computing device 104 retrieves a plurality of recipes 128 intended for a user refreshment effort. In an embodiment, recipes stored within recipe database may be organized by skill level and level of difficulty. In such an instance, computing device 104 may match a user refreshment effort to a recipe intended for the same and/or similar user refreshment effort. For instance and without limitation, a user refreshment effort that contains a description of a user who likes meals that are cooked and ready in one hour or less may be matched by computing device 104 to a plurality of recipes 128 that are intended for home cooks and that can be prepared in forty five minutes or less. In yet another non-limiting example, a user refreshment effort that contains a description of a user who likes to cook very gourmet and complex meals may be matched by computing device 104 to a alimentary style 128 that are complex and may take hours to prepare.

With continued reference to FIG. 4, computing device 104 adjusts recipes based on offered element available with a specified location of a user. Computing device 104 receives from a remote device 108 a user location. In another non-limiting example, a user location may contain a description of where a user will be traveling to and be located in upcoming months. Information pertaining to a user location may be contained within selection database 116. Computing device 104 identifies offered element contained within a user location. An offered element includes any recipe ingredient available for sale and/or purchase within the user location. For instance, and without limitation, an offered element may include an ingredient such as a Madagascar vanilla bean. Computing device 104 identifies if offered element are available within a user location. Information pertaining to availability of offered elements may be contained within recipe database. Computing device 104 may determine if an offered element is available, by generating a query and retrieving information pertaining to an offered element from within recipe database. Computing device 104 adjusts a recipe as a function of offered element contained within a user geolocation and an alimentary style 120. For instance, and without limitation, a user located in Bellingham, Washington may be unable to obtain a Madagascar vanilla bean. In such an instance, computing device 104 may consult recipe database and identify a substitution that can replace Madagascar vanilla bean and still comply with a user's alimentary style 120. In such an instance, computing device 104 may identify a second offered element that be substituted and available within the user's location, such as vanilla extract. In yet another non-limiting example, computing device 104 may identify an offered element such as fresh strawberries that are not available for a user within the user's location during winter months, when it is too cold to grow strawberries. In such an instance, computing device 104 adjusts a recipe to suggest frozen strawberries instead during the winter months when fresh strawberries are unavailable.

Additionally or alternatively, and still referring to FIG. 4, computing device may generate a recipe machine-learning model where the recipe machine-learning model utilizes the user selection as an input and outputs the plurality of recommended refreshments. In one embodiment, the recipe machine-learning model outputs a plurality of recommended refreshments as a function of the health of the user. In another embodiment, the recipe-machine learning model is trained using training data to select the plurality of recommended refreshments favored by the user selection. This may be implemented, without limitation, as described in FIGS. 1-3.

With continued reference to FIG. 4, at step 420, computing device may be configured to classify the plurality of recipes as a function of the health condition. Health alimentary classifier 132 is generated by using training data 136 correlating the health condition to the plurality of recipes and a classification algorithm as described herein. Computing device 104 receives the plurality of recipes and generates a refreshment instruction set as a function of the plurality of recipes and the health condition. This may be implemented, without limitations, as described in FIGS. 1-3. A refreshment instruction set 144 includes any of the refreshment instruction set 144 as described above in more detail in reference to FIG. 1. A refreshment instruction set 144 includes a meal plan containing recommended refreshments and a suggested temporal attribute. Computing device 104 generates a refreshment instruction set 144 by generating a recipe machine-learning model. Recipe machine-learning model includes any of the recipe machine-learning model as described above in more detail in reference to FIG. 1. Recipe machine-learning model utilizes a user selection 112 as an input and outputs recommended refreshments. Recipe machine-learning model is trained utilizing any of the training data as described above in more detail in reference to FIG. 1. Recipe machine-learning model is trained using training data to select recommended refreshments favored by a user selection 112. For instance and without limitation, one or more user selection 112 contained within selection database 116 may be utilized as training data to customized recipe machine-learning model to selected recommended refreshments favored by a user selection 112. For instance and without limitation, a user selection 112 that contains an entry specifying that a user enjoys consuming foods that contain ingredients such as black beans, grains, vegetables such as zucchini, carrots, and celery may be utilized to train recipe machine-learning model to output recommended refreshments containing those ingredients. Recommended refreshments include any of the recommended refreshments as described above in more detail in reference to FIG. 1. A recommended refreshment may include a recommended and/or suggested meal contained within a refreshment instruction set 144. A recommended refreshment may contain a suggested day and/or time when the recommended refreshment is suggested to be prepared and/or consumed. For example, a recommended refreshment may be suggested to be consumed for breakfast on a Friday or as an afternoon snack on a Wednesday. Computing device 104 assigns a recommended refreshment to a particular day and/or time based on identifying appointment information relating to a user. Appointment information includes any of the appointment information as described above in more detail in reference to FIG. 1. Computing device 104 may store appointment information relating to a user within selection database 116. Computing device 104 locates a constructed refreshment contained within a refreshment instruction set 144. A constructed refreshment includes any of the constructed refreshments as described above in more detail in reference to FIG. 1. Computing device 104 assigns a constructed refreshment on the day of an appointment. For instance, and without limitation, computing device 104 may identify an appointment relating to a user on a Wednesday afternoon, when a user will be occupied with a work meeting from 4 pm until 6 pm. In such an instance, computing device 104 locates a constructed refreshment contained within a refreshment instruction set 144, such as a meal of a premade roast chicken with a side salad. In such an instance, computing device 104 assigns the constructed refreshment containing the roast chicken and the side salad on the Wednesday afternoon when the user is occupied and will not have much time to cook and/or prepare dinner.

Additionally or alternatively, and with continued reference to FIG. 4, computing device may receive a user refreshment activity from the remote device and classify, by using a first classification algorithm, the user refreshment activity to an adherence level. Computing device may update the refreshment instruction set as a function of the adherence label. In an embodiment, the adherence label may include a numerical score. This may be implemented, without limitation, as describe in FIGS. 1-3.

With continued reference to FIG. 4, computing device may be configured to track the health of the user as a function of the adherence label of the user refreshment activity. This may be implemented, without limitation, as describe in FIGS. 1-3.

Still referring to FIG. 4, at step 425, computing device may output the refreshment instruction set. Computing device may identify an appointment relating to the user as a function of the schedule and locate a constructed refreshment contained within the refreshment instruction set. Computing device may assign the constructed refreshment on a day of the appointment. This may be implemented, without limitation, as described in FIGS. 1-3. A schedule includes any of the schedules as described above in more detail in reference to FIG. 1. A schedule may contain one or more appointments pertaining to a user, including any of the appointments as described above in more detail in reference to FIG. 1. In an embodiment, a schedule may contain a series of appointments scheduled for the next six weeks or a given period of time. In an embodiment, computing device 104 receives an updated schedule such as when appointments are changed or scheduled at different times, utilizing any network methodology as described herein.

Alternatively or additionally, computing device may transmit the refreshment instruction set to the remote device and receive a user response rejecting the refreshment instruction set. Computing device 104 may transmit a second refreshment instruction set to the remote device.

With continued reference to FIG. 4, computing device may transmit a communication comprising information as a function of the health condition of the user and the refreshment instruction set. This may be implemented, without limitation, as described in FIGS. 1-3.

With continued reference to FIG. 4, computing device is configured to receive a user refreshment activity from a remote device. A user refreshment activity includes any of the user refreshment activities as described above in more detail in reference to FIG. 1. A user refreshment activity may include for example, a list of meals that a user ended up cooking and eating during the time period contained within a refreshment instruction set 144. Computing device 104 classifies, using a first classification algorithm, a user refreshment activity to an adherence label. An adherence label includes any of the adherence label as described above in more detail in reference to FIG. 1. An adherence label may contain an indication as to how well a user refreshment activity complied with a refreshment instruction set 144. For instance and without limitation, a user refreshment activity that specifies a user has not prepared and/or cooked any meals as contained within a refreshment instruction set 144 may be classified to an adherence label that describes the user as being noncompliant. Computing device 104 updates a refreshment instruction set 144 utilizing an adherence label. For example, an adherence label that specifies a user has been moderately adherent may be utilized to update a refreshment instruction set 144 to recommend refreshments that can be cooked and/or prepared in less time or that may require less skill. In yet another non-limiting example, an adherence label that specifies a user has been extremely compliant may be utilized to generate recipe recommendations that are of similar complexity and require similar preparations and/or ingredients. In yet another non-limiting example, an adherence label that specifies a user has been moderately adherent may be utilized to update a refreshment instruction set 144 to select recommended refreshments that may be less complicated, and easier to prepare. In an embodiment, computing device 104 may prompt a user with one or more questions to determine what prevented a user from being completely adherent. In such an instance, computing device 104 may transmit to remote device one or more questions to evaluate and understand what happened and/or what prevented the user from being more compliant with a refreshment instruction set 144. Such information and/or responses from a user may be utilized to recommend refreshments that contain less complicated ingredients, are easier to prepare, require less skill or sophistication, require more basic culinary equipment and the like.

With continued reference to FIG. 4, computing device creates a list of elements necessary to create a plurality of recommended refreshments. A list includes any of the lists as described above in more detail in reference to FIG. 1. A list contains a compilation of elements needed to prepare and cook recommended refreshments contained within a refreshment instruction set 144. Computing device 104 may create a list of elements and receive user feedback containing an input and/or selection as to which elements the user may already have at home. In such an instance, computing device 104 updates a list of elements to contain only those elements that need to be purchased by a user. Computing device 104 categorizes a list of elements offered at refreshment providers. Categorization includes sorting elements based on where a user can purchase elements at various refreshment locations. Computing device 104 may retrieve information pertaining to refreshment providers that a user shops at and purchases elements from stored in selection database 116. For instance and without limitation, a list of elements may be categorized to contain a first list of elements such as pantry staples that a user can purchase from an online virtual market, and a second list of elements such as fresh produce and ethically raised meat that a user can purchase from a farm stand down the street from a user's house. In such an instance, computing device 104 categorizes elements to aggregate elements that a user needs to purchase at a first refreshment provider and elements that a user needs to purchase at a second refreshment provider.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

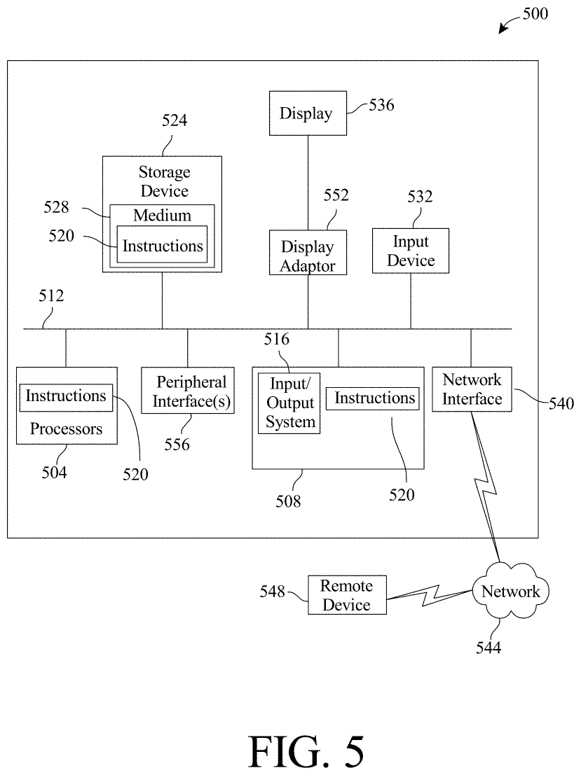

FIG. 5 shows a diagrammatic representation of one embodiment of a computing device in the exemplary form of a computer system 500 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 500 includes a processor 504 and a memory 508 that communicate with each other, and with other components, via a bus 512. Bus 512 may include any of several types of bus structures including, but not limited to, a memory bus, a memory controller, a peripheral bus, a local bus, and any combinations thereof, using any of a variety of bus architectures.

Processor 504 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 504 may be organized according to Von Neumann and/or Harvard architecture as a non-limiting example. Processor 504 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC)

Memory 508 may include various components (e.g., machine-readable media) including, but not limited to, a random-access memory component, a read only component, and any combinations thereof. In one example, a basic input/output system 516 (BIOS), including basic routines that help to transfer information between elements within computer system 500, such as during start-up, may be stored in memory 508. Memory 508 may also include (e.g., stored on one or more machine-readable media) instructions (e.g., software) 520 embodying any one or more of the aspects and/or methodologies of the present disclosure. In another example, memory 508 may further include any number of program modules including, but not limited to, an operating system, one or more application programs, other program modules, program data, and any combinations thereof.

Computer system 500 may also include a storage device 524. Examples of a storage device (e.g., storage device 524) include, but are not limited to, a hard disk drive, a magnetic disk drive, an optical disc drive in combination with an optical medium, a solid-state memory device, and any combinations thereof. Storage device 524 may be connected to bus 512 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 524 (or one or more components thereof) may be removably interfaced with computer system 500 (e.g., via an external port connector (not shown)). Particularly, storage device 524 and an associated machine-readable medium 528 may provide nonvolatile and/or volatile storage of machine-readable instructions, data structures, program modules, and/or other data for computer system 500. In one example, software 520 may reside, completely or partially, within machine-readable medium 528. In another example, software 520 may reside, completely or partially, within processor 504.

Computer system 500 may also include an input device 532. In one example, a user of computer system 500 may enter commands and/or other information into computer system 500 via input device 532. Examples of an input device 532 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 532 may be interfaced to bus 512 via any of a variety of interfaces (not shown) including, but not limited to, a serial interface, a parallel interface, a game port, a USB interface, a FIREWIRE interface, a direct interface to bus 512, and any combinations thereof. Input device 532 may include a touch screen interface that may be a part of or separate from display 536, discussed further below. Input device 532 may be utilized as a user selection device for selecting one or more graphical representations in a graphical interface as described above.

A user may also input commands and/or other information to computer system 500 via storage device 524 (e.g., a removable disk drive, a flash drive, etc.) and/or network interface device 540. A network interface device, such as network interface device 540, may be utilized for connecting computer system 500 to one or more of a variety of networks, such as network 544, and one or more remote devices 548 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 544, may employ a wired and/or a wireless mode of communication. In general, any network topology may be used. Information (e.g., data, software 520, etc.) may be communicated to and/or from computer system 500 via network interface device 540.

Computer system 500 may further include a video display adapter 552 for communicating a displayable image to a display device, such as display device 536. Examples of a display device include, but are not limited to, a liquid crystal display (LCD), a cathode ray tube (CRT), a plasma display, a light emitting diode (LED) display, and any combinations thereof. Display adapter 552 and display device 536 may be utilized in combination with processor 504 to provide graphical representations of aspects of the present disclosure. In addition to a display device, computer system 500 may include one or more other peripheral output devices including, but not limited to, an audio speaker, a printer, and any combinations thereof. Such peripheral output devices may be connected to bus 512 via a peripheral interface 556. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. A system for generating a refreshment instruction set, the system comprising:
a computing device, the computing device configured to:
receive, from a remote device, a user selection, the user selection identifying a nourishment and a health condition of a user;
determine an alimentary style relating to the user selection using an alimentary style classifier, wherein the alimentary style classifier inputs the nourishment and outputs the alimentary style;
retrieve a plurality of recipes relating to the alimentary style;
classify the plurality of recipes as a function of the health condition, wherein classifying comprises:
receiving the plurality of recipes;
generating, using training data correlating the health condition to the plurality of recipes and a classification algorithm, a health alimentary classifier; and
determining a refreshment instruction set as a function of the health condition and the health alimentary classifier; and
output the refreshment instruction set wherein outputting the refreshment instruction set comprises:
identifying an appointment relating to the user as a function of the schedule;
locating a constructed refreshment contained within the refreshment instruction set; and
assigning the constructed refreshment on a day of the appointment.

2. The system of claim 1, wherein the computing device is further configured to train the alimentary style classifier using training data, wherein the training data correlates the health condition to a corresponding alimentary style.

3. The system of claim 1, wherein the computing device is further configured to generate a recipe machine-learning model, wherein the recipe machine-learning model utilizes the user selection as an input and outputs the plurality of recommended refreshments.

4. The system of claim 3, wherein the recipe machine-learning model outputs a plurality of recommended refreshments as a function of the health condition of the user.

5. The system of claim 3, wherein the recipe machine-learning model is trained using training data to select the plurality of recommended refreshments favored by the user selection.

6. The system of claim 1, wherein the computing device is further configured to:
receive a user refreshment activity from the remote device;
classify, using a first classification algorithm, the user refreshment activity to an adherence label; and
update the refreshment instruction set as a function of the adherence label.

7. The system of claim 6, wherein the adherence label comprises a numerical score.

8. The system of claim 6, wherein the computing device is further configured to track the health condition of the user as a function of adherence label of the user refreshment activity.

9. The system of claim 1, wherein the computing device is further configured to:
transmit the refreshment instruction set to the remote device;
receive, from the remote device, a user response rejecting the refreshment instruction set; and
transmit a second refreshment instruction set to the remote device.

10. The system of claim 1, wherein the computing device is further configured to transmit a communication comprising information as a function of the health condition of the user and the refreshment instruction set.

11. A method for generating a refreshment instruction set, the method comprising:
receiving, from a remote device, a user selection as a function of at least nourishment or health condition of a user;
determining, by a computer device, an alimentary style relating to the user selection using an alimentary style classifier, wherein the alimentary style classifier utilizes the user selection as a function of at least nourishment or health condition as an input, and outputs the alimentary style.
retrieving, by the computer device, a plurality of recipes relating to the alimentary style;
classifying, by the computer device, the plurality of recipes as a function of the health condition, wherein classifying comprises:
receiving the plurality of recipes;
generating using training data correlating the health condition to the plurality of recipes and a classification algorithm, a health alimentary classifier; and
determining a refreshment instruction set as a function of the health condition and the health alimentary classifier; and
outputting, by the computer device, the refreshment instruction set wherein outputting the refreshment instruction comprises:
identifying an appointment relating to the user as a function of the schedule;
locating a constructed refreshment contained within the refreshment instruction set; and
assigning the constructed refreshment on a day of the appointment.

12. The method of claim 11, further comprising training the alimentary style classifier using training data, wherein the training data correlates a health condition to a corresponding alimentary style.

13. The method of claim 11, further comprising generating a recipe machine-learning model, wherein the recipe machine-learning model utilizes the user selection as an input and outputs the plurality of recommended refreshments.

14. The method of claim 13, wherein the recipe machine-learning model outputs a plurality of recommended refreshments as a function of the health of the user.

15. The method of claim 13, wherein the recipe machine-learning model is trained using training data to select the plurality of recommended refreshments favored by the user selection.

16. The method of claim 11, further comprising:
receiving a user refreshment activity from the remote device;
classifying, using a first classification algorithm, the user refreshment activity to an adherence label; and
updating the refreshment instruction set as a function of the adherence label.

17. The method of claim 16, wherein the adherence label comprises a numerical score.

18. The method of claim 16, wherein the computing device is further configured to track the health of the user as a function of adherence label of the user refreshment activity.

19. The method of claim 11, further comprising:
transmitting the refreshment instruction set to the remote device;
receiving, from the remote device, a user response rejecting the refreshment instruction set; and
transmitting a second refreshment instruction set to the remote device.

20. The method of claim 11, further comprising transmitting a communication comprising information as a function of the health condition of the user and the refreshment instruction set.
